(19) United States
(12) Patent Application Publication
(10) Pub. No.: US 2014/0155356 A1
(43) Pub. Date: Jun. 5, 2014

(54) CURABLE BONE SUBSTITUTE

(71) Applicant: A Enterprises, Inc., (US)

(72) Inventors: Arthur Ashman, Westport, CT (US); Devon A. Shipp, Potsdam, NY (US)

(73) Assignee: A Enterprises, Inc., Westport, CT (US)

(21) Appl. No.: 13/690,562

(22) Filed: Nov. 30, 2012

(51) Int. Cl. A61L 27/54; A61L 27/00; A61L 27/56

(52) U.S. Cl. CPC A61L 27/54; A61L 27/56; A61L 27/00
USPC 514/152; 623/23.5; 623/23.59; 623/23.58

(57) ABSTRACT

A novel composition, kit, and method of using the composition as a bone substitute for dental, orthopedic and drug delivery purposes. Specifically, the bone substitute comprises a plurality of polymeric beads having a crosslinkable shell where the shell is cured by light and/or chemical curing.

This application claims priority to U.S. provisional application 60/728,670 filed Oct. 19, 2005, herein incorporated by reference.

### FIELD OF THE INVENTION

The present invention relates generally to materials which may be used in any part of the body as an implant or graft material. More particularly, it relates to porous implants which allow for the growth of bone and gum tissue into the implant to assure that it is firmly attached to the body structures and becomes an integral part or fixation thereof.

### BACKGROUND OF THE INVENTION

In the healing arts, there is often a need for an implant or graft material to replace, repair, or reconstruct tissues, in particular, hard tissues such as bone. For example, hard-tissue implant materials have been used in medicine and veterinary medicine as prosthetic bone materials to repair injured or diseased bone. Hard tissue implant materials are also used in the construction of prosthetic joints to fix the prosthetic joints to bones. In the dental art, hard tissue implant materials are used in the reconstruction of jaw bone damages caused by trauma, disease, or tooth loss; in the replacement or augmentation of the edentulous ridge; in the prevention of jaw bone loss by socket grafting; and in the treatment of periodontal bone void defects.

Specifically, in the dental art, when a tooth is extracted, a large cavity is created in the alveolar bone. The alveolar bone begins to undergo resorption at a rate of 40-60% in 2-3 years, which continues yearly at a rate of 0.25% to 0.50% per year until death (Ashman A. et al., Prevention of Alveolar Bone Loss Post Extraction with Bioplant® HTR® Grafting Material. Oral Surg. Oral. Med. Oral. Pathol. 60 (2):146-153, (1985)). Shifting of the remaining teeth, pocket formation, bulging out of the maxillary sinus, poor denture retention, loss of vertical dimension, formation of facial lines, unaesthetic gaps between bridgework and gum are some of the undesirable consequences associated with such loss (Luc. W. J. Huys, Hard Tissue Replacement, Dentist News, (Feb. 15, 2002)). Such bone loss also creates a significant problem for the placement of dental implants to replace the extracted tooth. It has been reported in previous years that nearly 95% of implant candidates rejected were attributable to inadequate height and/or width of the alveolar bone (Ashman A., Ridge Preservation, Important Buzzwords in Dentistry, General Dentistry, May/June, (2000)).

One proven technique for overcoming the bone and soft tissue problems associated with the extraction of the tooth is to fill the extraction site with a bone graft material (e.g., synthetic, bovine or cadaver derived), and cover the site with gum tissue (e.g., suturing closed) or a dental “bandage” (e.g., Biofoil® Protective Stripes) for a period of time sufficient for new bone growth. The cavity becomes filled with a mixture of the bone graft material acting as an osteoconductive scaffold for the newly regenerated/generated bone. When implant placement is desired, after a period of time sufficient to allow bone regeneration (or healing) in the cavity, a cylindrical bore drill can prepare the former extraction site, and a dental implant can be installed in the usual manner.

U.S. Pat. No. 4,199,864 discloses a method for fabricating polymeric plastic implants for endosteal or periosteal applications having porous surfaces with pores of a predetermined size, pore depth, and degree of porosity. Leachable substances, such as sodium chloride crystals of controlled particle size are added to a powdered polymer-liquid monomer mixture in proportional amounts corresponding to the desired degree of porosity. These crystals, combined with mold release agents, are used to coat mold cavity surfaces to achieve proper near-surface porosity. After heat polymerization without the use of an initiator, and abrasive removal of resulting surface skin, the salt is removed by leaching to provide the desired porosity. Bone ingrowth is promoted by pore sizes in the 200-400 micron range. Pore sizes of 50-150 microns result in soft tissue ingrowth.

U.S. Pat. Nos. 4,535,485 and 4,536,158 disclose certain implantable porous prostheses for use as bone or other hard tissue replacement which are comprised of polymeric particles. The particles have a core comprised of a first biologically-compatible material such as polymethylmethacrylate and a coating comprised of a second biologically-compatible polymeric material which is hydrophilic, such as polymeric hydroxyethylmethacrylate. The particles may incorporate a radio-opaque material to render the particle visible in an X-ray radiograph. The mass of the particles may be implanted in the body in a granulate form. The interstices between the implanted particles form pores (i.e., extra-particle pores) into which tissue can grow. The hydrophilic coating on the particles facilitates infusion of body fluids into the pores of the implant, which facilitates the ingrowth of tissue into the pores of the implant.

U.S. Pat. No. 4,728,570 discloses a porous implant material which induces the growth of hard tissue. Based on the '570 patent, Bioplant Inc. (South Norwalk, Conn.) manufactures a slowly absorbable product called Bioplant® HTR®. This product has proven to be very useful in both preventing bone loss and stimulating bone generation. It has also been found suitable for esthetic tissue plumping as well as for immediate post-extraction implants as mentioned above. However, like bone graft materials prior to the present invention, when placed in an extraction socket or in edentulous spaces, the implant would not be immediately functional. A patient still must wait months for bone generation (e.g., osteointegration) to take place around the implant before revisiting the dentist's office months later to have a crown installed.

Therefore, there is a continued need in the replacement and restorative arts for materials and methods which reduce the time of the bone regenerative process, allow immediately functional dental implants, provide sufficient mechanical strength, and/or minimize micro-movement. In addition, there is a need to broaden the spectra of materials available for dental and orthopedic implants. There is also need for materials that can also be used for the delivery of drugs or other active agents to the surrounding tissue.

### SUMMARY OF THE INVENTION

The present invention provides a crosslinkable bone substitute comprising a porous biologically compatible material. More specifically, the crosslinkable bone substitute comprises polymer beads, physically coated with a second polymeric material, the “shell polymeric material” that is hydrophilic in nature. Furthermore, the shell material comprises at least a crosslinkable reactive group.

The foregoing invention provides a crosslinked bone substitute comprising a plurality of crosslinked coated polymer beads, where the crosslinking groups link the shell to the shells of other polymer beads. The invention also provides a bone substitute which immediately hardens upon crosslinking and becomes load-bearing. In particular, upon crosslinking, the bone substitute provides for a composite with homogeneous mechanical properties and, concomitantly, a high level of structural and mechanical integrity.

In the present invention, the crosslinkable bone substitute is an alloplast. Preferably, the crosslinkable bone substitute comprises a polymer alloplast. More preferably, the polymer alloplast (porous or non-porous) comprises a core layer comprised of a first polymeric material, the “core polymeric material” and a shell generally surrounding the core layer comprising a second monomeric or polymeric material, the “shell polymeric material,” wherein the shell material is hydrophilic. The core and shell polymeric materials are biocompatible, and comprise different compositions. Preferably, the crosslinkable bone substitute comprises porous micron-sized particles; preferably, the diameter is in the range of from about 250 microns to about 900 microns.

In preferred embodiments, the core polymeric material of the bone substitute comprises polymethylmethacrylate (PMMA) and polymeric hydroxyethylmethacrylate (PHEMA)

The shell polymeric material is a hydrophilic substrate that is biocompatible, non-toxic, and contains reactive groups that can react to create a polymeric network and is preferably a polyethylene glycol (PEG), HEMA or modified HEMA.

A crosslinkable reactive group comprises a polymerizable group characterized by its ability to crosslink to form a polymer network. The crosslinking may be electrostatic or chemical in nature. Some preferred crosslinking groups include ethylenes, carbonyls, alcohols, esters, amines, amides, etc.

The crosslinkable bone substitute is crosslinked by an initiator, preferably a photoinitiator, a redox initiator, or a combination of a photoinitiator and a redox initiator system.

Optionally, the composition further comprises a therapeutic agent, a bone promoting agent, a porosity forming agent, and/or a diagnostic agent.

The crosslinkable bone substitute and the crosslinked composite are useful in the field of orthopedics and dentistry. They can be used anywhere where bone or other tissue regeneration is required. When a therapeutic agent is incorporated in them, they are additionally useful for the controlled delivery of the therapeutic agents as well (i.e., promoting bone growth by the slow release of a bone growth protein or limiting infection by the slow release of an antiviral agent.)

### DESCRIPTION OF THE DRAWINGS

FIGS. 1A, 1B, and 1C. Stress-strain diagrams for cured samples. FIG. 1A, HTR: PEG-DM (82/18). FIG. 1B, HTR:HEMA (80/20). FIG. 1C HTR:PEG-DM/HEMA (10% w/w).

FIGS. 2A, 2B, and 2C. SEM micrographs of Bioplant® HTR®:PEG-DM.

FIGS. 3A, 3B, and 3C. SEM micrographs of Bioplant® HTR®:HEMA.

FIGS. 4A, 4B, and 4C. SEM micrographs of Bioplant® HTR®:EG-DM/HEMA.

FIGS. 5A, 5B, and 5C. SEM micrographs of Bioplant® HTR®:PEG-DM showing surface morphology after compression testing.

FIGS. 6A, 6B, and 6C. SEM micrographs of Bioplant® HTR®:HEMA showing surface morphology after compression testing.

FIGS. 7A, 7B, and 7C. SEM micrographs of Bioplant® HTR®:EG-DM/HEMA showing surface morphology after compression testing.

### DETAILED DESCRIPTION OF THE INVENTION

The present invention relates to crosslinkable bone substitute comprising a core biologically compatible material surrounded by a second polymeric or polymerizable material, and to a crosslinked bone substitute or composite where the second polymeric or polymerizable material is polymerized to harden around the core material. The present invention also relates to methods of forming and using the crosslinked bone substitute or composite.

The crosslinked bone substitute comprises a plurality of crosslinked coated polymer beads, wherein the coated polymer beads comprise a core polymeric material and a shell polymeric material. The crosslinked bone substitute is formed by mixing a plurality of coated polymer beads, wherein the crosslinkable reactive groups of the shell polymeric material crosslink via chemical or electrostatic bonds to form a substantially homogeneous mixture.

As used herein, the following polymer abbreviations are used:

Bioplant® HTR® microporous particles of calcified (Ca(OH)₂/calcium-carbonate) copolymer of PMMA and PHEMA
CPP 1,3-bis(p-carboxyphenoxy) propane
CPP-SA 1,3-bis(p-carboxyphenoxy) propane—sebacic acid copolymer
DMAEMA 2-Dimethylaminoethyl methacrylate
HA hydroxyapatite
HEMA 2-Hydroxyethyl methacrylate
LDPE low density polyethylene
MCPP methacrylated p-carboxyphenoxypropane
MMA methyl methacrylate
mPEG modified poly(ethylene glycol)
MSA methacrylated sebacic acid
NVP N-vinyl pyrrolidone
PHEMA polymeric hydroxyethylmethacrylate
PEG poly(ethylene glycol)
PEG-DA poly(ethylene glycol)dimethacrylate
PEG-MA poly(ethylene glycol) methacrylate
PGA poly(glycolic acid)
PLA poly(lactic acid)
PMMA poly(methyl methacrylate)
PVP polyvinyl pyrrolidone
TCP tricalcium phosphate

Other common abbreviations utilized herein include:

3-DMAB 3-dimethylaminobenzoic acid
4-DMAB 4-dimethylaminobenzoic acid
4-EDMAB 4-ethyl p-dimethylaminobenzoate
EG-DA (ethylene glycol)dimethacrylate
AIBN azoisobutyronitrile

### BPO Benzoyl Peroxide

CQ camphorquinone
DHEPT N,N-bis(2-hydroxyethyl)-p-toluidine
DMABA 4-dimethylaminobenzoate
DMAPE 4-dimethylaminophenethanol

### DMPT-N,N-dimethyl-p-toluidine

### EA Ethyl Acetate

EDMAB ethyl p-dimethylaminobenzoate
EHDMAB 2-ethylhexyl p-dimethylaminobenzoate
Irgacure 651® 2,2-dimethoxy-2-phenylacetophenone
T-BDMA 4-t-butyl dimethylaniline
TEA triethylamine

### I. Core Bone Substitute Materials

The core material of the crosslinkable bone substitute is a biologically compatible material that contains calcium on the surface of the material. It forms a hard material that does not produce a toxic, injurious, or immunological response in living tissue such as blood, bones, and gums. Preferably, the crosslinkable bone substitute comprises an alloplast. By “alloplast” is meant a synthetic bone substitute. Non-limiting examples of the alloplast include calcium phosphate and calcium sulfate ceramics and polymeric bone graft materials.

The alloplast is preferably a plurality of micron-sized particles. As used herein, the phrase “micron size” indicates the size is on a micron scale including 1-1000 μm, or more particularly 400-900 μm or more particularly 600-800 μm, each particle comprising a core polymeric material. Preferably, the polymeric material is biocompatible. The core polymeric material is preferably one or more acrylic polymers; more preferably, PHMMA or PHEMA, or a combination thereof. The core material may further include a plasticizer, if desired.

In one embodiment, preferred polymeric particles are similar to those disclosed in the '485 patent, the specification of which is hereby incorporated by reference in its entirety.

In one preferred embodiment, the core bone substitute is a plurality of calcium hydroxide-treated polymeric micron-sized particles. The quantity of calcium hydroxide is effective to induce the growth of hard tissue in the pores and on the surface of the polymeric micron-sized particles when packed in a body cavity, preferably in amounts of from 1 to 30 weight % of the bone substitute. Preferably, the calcium hydroxide forms a coating on both the outer and interior surfaces (pores) of the polymeric particles.

The micron-sized particles of the bone substitute may further optionally include an agent that is radio-opaque to render the bone substitute visible on an X-ray radiograph.

Preferred procedures for producing the bone substitute of the present invention are set forth in the specification of the '158 patent.

In a most preferred embodiment, the bone substitute is an improved curable form of Bioplant® HTR®. The original form of Bioplant® HTR® is set forth in the '570 patent, which is hereby incorporated by reference in its entirety. The improved form of Bioplant® HTR® comprises particles of calcified (Ca(OH)₂/calcium-carbonate) copolymer of PMMA and PHEMA, with the outer calcium layer interfacing with bone forming calcium carbonate-apatite. Bioplant® HTR® has pores within the particles (inter-particle pores) into which tissue can grow. The outer diameter of the particles is about 750 μm; the inner diameter is about 600 μm and the pore opening diameter is about 350 μm. When packed in place, interstices form between the implanted Bioplant® HTR® particles form pores (i.e., extra-particle pores) into which tissue can grow. Bioplant® HTR® is strong (forces greater than 50,000 lb/in will not crush the Bioplant® HTR® particles), biocompatible and negatively charged (−10 mV) to promote cellular attraction and resist infection.

In another embodiment, the biocompatible polymeric material is a calcium phosphate material such as hydroxyapatite (HA), tricalcium phosphate (TCP), or a mixture or hybrid thereof.

Hydroxyapatite, (Ca₁₀(PO₄)₆(OH)₂) is one of the most biocompatible materials with bones; it is naturally found in bone mineral and in the matrix of teeth and provides rigidity to bones and teeth. When a HA-containing material is used as a bone substitute in the present invention, the modulus will be significantly increase. A non-limiting list of HA bone substitutes that may be used in the present invention include: Pro Osteon® (Interpore Cross International, Inc., Irvine, Calif.) comprising monolithic ceramic granules, which are made using coralline calcium carbonate fully or partially converted to HA by a hydrothermal reaction, see D. M. Roy and S. K. Linnehan, Nature, 247, 220-222 (1974); R. Holmes, V. Mooney, R. Bucholz and A. Tencer, Clin. Orthop. Rel. Res., 188, 252-262 (1984); and W. R. Walsh, et al., J. Orthop. Res., 21, 4, 655-661 (2003). VITOSS® (Orthovita, Malvern, Pa.) is provided as monolithic ceramic granules. Norian SRS® (Synthes-Stratec, affiliates across Europe and Latin America) and Alpha-BSM® (ETEX Corp., Cambridge, Mass.) are provided as an injectable pastes. ApaPore® and Pore-SI (ApaTech, London, England) are currently under development and comprise monolithic ceramic granules.

Other HA bone substitutes that may be used included in the bone substitute of the present invention is resorbable carbonated apatite. One particularly preferred HA, is a porous calcium phosphate material which is a porous hydroxyapatite and is more integrable, absorbable and more osteoconductive than dense hydroxyapatite. Porous HA can be made by the methods described in EP1411035, herein incorporated by reference. The aporosity can be controlled both as a ratio of the volume of material to the volume of air and as the porosity and pore size distribution.

Additionally, recent studies have elucidated the detrimental and beneficial effects of minor amounts of impurities and some dopants. Parts per million levels of lead, arsenic, and the like, if incorporated into hydroxyapatite, may lead to inhibition of osteoconduction. It is therefore preferable to use HA substantially free from these impurities. On the other hand, carbonated apatite exhibits faster bioresorption than pure HA, if desired, and 1-3 wt % silicon additions to HA have shown a two-fold increase in the rate of osteoconduction over pure HA, see N. Patel, et al., J. Mater. Sci: Mater. Med., 13, 1199-206 (2002); and A. E. Portera, et al., Biomaterials, 24, 4609-4620 (2002). Silicon-doped HA such as the doped HA being developed at ApaTech and may be used as a filler in the present invention.

In one embodiment, the filler is preferably a calcium phosphate material based upon HA, including alpha (α-TCP) or beta-tricalcium phosphate (Ca3(PO4)2, α-TCP), which is a close synthetic equivalent to the composition of human bone mineral and has favorable resorption characteristics.

α-TCP has a high resorbability when the material is implanted in a bone defect and is sold as Biosorb®. Other calcium phosphates including biphasic calcium phosphate or BCP (an intimate mixture of HA and α-TCP) and unsintered apatite (AP) may also be used as bone substitutes in the present invention.

In another embodiment, the TCP material may be a TCP having a particularly small crystal size and/or particle size. This TCP (i.e., α- and/or β-TCP) is formed into high surface area powders, coatings, porous bodies, and dense articles by a wet chemical approach and transformed into TCP, for example by a calcination step such as that described in U.S. Pat. Pub. 2005/0031704, herein incorporated by reference. This TCP material, generally having an average TCP crystal size of about 250 nm or less and an average particle size of about 5 μm or less, has greater reliability and better mechanical properties as compared to conventional TCP having a coarser microstructure and is therefore one particularly preferred embodiment of the present invention.

Also useful for incorporation with the biologically compatible polymeric materials are biologically compatible cadaver bone and bovine bone materials. These materials may be mixed with the polymeric material to form the core material.

When calcium hydroxide is added to the core material, upon exposure to aqueous solution (e.g., blood), the calcium hydroxide on the core bone substitute is converted to a calcium carbonate apatite (bone) compound. Preferably, calcium hydroxide is introduced into the pores of the micron-sized particles by soaking the particles in an aqueous solution of calcium hydroxide, then removing any excess solution from the particles and allowing the particles to dry. Preferred aqueous solutions of calcium hydroxide have a concentration in the range of from about 0.05 percent to about 1.0 percent calcium hydroxide by weight.

### II. The Shell Polymeric Material

The shell material is a polymer or polymerizable material that is biocompatible, non-toxic, and contains reactive groups that can react to create a polymeric network (e.g., a polymer or a prepolymer). The monomers and/or prepolymers are required to coat the surface of the synthetic bone substitute, and upon curing form a hard polymeric network. The shell will contain a group capable of polymerizing such as a vinyl group, cyclic ester, or a difunctional group such as a diamine and diacids. Typical examples of monomers are HEMA, PEG-MA, PEG-DM, DMAEMA, and NVP. In one preferred embodiment, the monomer/prepolymer coating preferably will consist of at least one component containing more than one vinyl group to ensure crosslinking occurred. In one preferred embodiment, the polymer is a hydrophilic polymer having one or more vinyl group.

Several non-limiting examples of polymeric coating materials are PEG, PHEMA, and modified PHEMA.

### Poly (Ethylene Glycol) Shell

The PEG polymer used to coat the core bone substitute can be linear, branched, or star-shaped with a wide range of molecular weight.

PEG dimethacrylate is one particularly preferred coating material. Different molecular weights of this polymer are contemplated, such as PEG-DM 100, PEG-DM 300, PEG-DM 600, and PEG-DM 1000. The difunctionallity creates a crosslinked network between the PEG on one particle and the PEG shell on other particles. Different molecular weight PEGs can be used to provide different viscosities and thereby effect the mixing, shell material thickness, density and polarity.

PEG methacrylate is another polymer that may be used for the shell polymeric material.

Additional PEG reagents that may also be used in various shell component embodiments include carboxyl-PEGs, esters-PEGs, aldehyde-PEGs (e.g., —CH2CH2-CHO), amino-PEGs (e.g., —CH₂CH₂CH₂NH₂ or —CH₂CH₂NH₂), acetal-PEGs (e.g., —CH2CH2CH(OC2H5)2), tresyl-PEGs (e.g., SO₂CH₂CF₃), thiol-PEGs (e.g., —CH2CH2SH), maleimido-PEGs (e.g., —CH₂CH₂CH₂NHCOCH₂CH₂-Maleimide or —CH₂CH₂CH₂-Maleimide), —CO₂-phenyl-NO₂—PEG, functionalized PEG-phospholipid, and other similar and/or suitable reactive PEGs as selected by those skilled in the art for their particular application and usage.

Poly (Hydroxyethyl Methacrylate) Shell

PHEMA, a polymer that is more flowable and more hydrophilic than PEG may, alternatively, be used as a shell material.

This coating is particularly useful when flowability is important, such as when delivery via a syringe is used (e.g., a HEMA and Bioplant® HTR® mixture is combined with an initiator within a syringe, then delivered directly to the area in need of a bone substitute and then cured.)

N-Vinyl Pyrrolidone Shell

N-vinyl pyrrolidone (NVP), which polymerizes to form poly(vinyl pyrrolidone) (PVP, povidone) is a commonly used biocompatible polymer and may be used as the shell material. The NVP can coat the core bone substitute and will polymerize to create a crosslinked PVP shell around the core.

### DMAEMA Shell

2-Dimethylaminoethyl methacrylate (DMAEMA) may also be used to form the crosslinkable shell in the present invention.

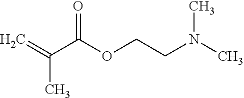

The DMAEMA can also be used to coat the core material and form a crosslinked shell.

Additional Shell Materials

Other materials useful as a crosslinkable shell material include methacrylic monomers such as triethyleneglycol dimethacrylate (generally used as a cross-linking agent for adhesives and dental restorative materials); urethane dimethacrylate, a methacrylate based on a methacrylated aliphatic isocyanate and used in dental bonding agents, resin veneering and restorative materials; 1,4-butanediol dimethacrylate, a cross-linking methacrylate monomer, which has also been used in dental composites, sealants and proteses; 2,2-bis(4-(2-hydroxy-3-methacryloxypropoxy)phenylpropane) (BIS-GMA, used as a dental composite restorative materials and dental sealants); and 2,2-bis(4-(methacryloxy)phenyl)propane (BIS-MA) which is a bisphenol-based monomer used in dental restorative composites and adhesive materials.

Acrylic monomers may also be used in the shell material. These compounds include, but are not limited to: 2-hydroxymethacrylate (commonly used in UV-inks, adhesives, lacquers and artificial nails) and 1,6-hexanediol diacrylate (commonly used in UV-cured inks, adhesives, coatings, photoresiting, castings and artificial nails).

The shell materials may be used individually or as copolymers (block, alternating, or random copolymers). Particular copolymers include a copolymer of NVP and DMAEMA, a copolymer of PEG-DM and PHEMA, or a copolymer of PHEMA and NVP.

### III. Initiators

The present invention utilizes an initiator system to cure the crosslinkable prepolymer. In one embodiment, both light curing and chemical curing is used. The initiator system is divided into two parts, the first part (component A) comprising the light and chemical initiators and the second part (component B) comprising the light and chemical accelerators. This system allows for fast curing of the polymer from light curing, while the chemical curing initiates the cross-linking reaction throughout the polymer matrix and increases the viscosity so that the material sets homogeneously.

In another embodiment, only chemical curing is used. Therefore, the initiator system comprises component A having a chemical initiator and component B comprising a chemical accelerator.

In one preferred embodiment, the two initiator components are mixed with the crosslinkable prepolymer immediately before curing. In other embodiments, one of the components is mixed with a component of the polymer or monomer or with the filler component prior to curing (e.g. to form a kit that can be easily manipulated to crosslink the prepolymer. When the initiator is pre-mixed, care must be taken to combine components so as not to degrade the polymer or prepolymer (particularly where the polymer is an anhydride which can be unstable in the presence of an oxidant) or destroy the initiator.

Initiator Component A

In a first embodiment, component A comprises a radical generating photoinitiator activated by electromagnetic radiation. This may be ultraviolet light (e.g., long wavelength ultraviolet light), light in the visible region, focused laser light, infra-red and near-infra-red light, X-ray radiation or gamma radiation. Preferably, the radiation is light in the visible or UV region and, more preferably, is blue light or UV light. Exposure of the photoinitiator and a co-catalyst such as an amine to light generates active species. Light absorption by the photoinitiator causes it to assume a triplet state; the triplet state subsequently reacts with the co-catalyst to form an active species which initiates polymerization.

Non-limiting examples of the photoinitiators include biocompatible photoinitiators such as beta carotene, riboflavin, Irgacure 651® (2,2-dimethoxy-2-phenylacetophenone), phenylglycine, dyes such as erythrosin, phloxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, and other acetophenone derivatives, and camphorquinone. A preferred photoinitiator is camphorquinone.

Component A also comprises a second free radical generator. The free radical generator is an oxidizing agent (also called an oxidizing component), such as peroxide. This agent is combined in a redox couple by mixing component A with component B, resulting in the generation of an initiating species (such as free radicals, anions, or cations) capable of causing curing. Preferably, the redox couples of this invention are activated at temperatures below about 40° C., for example, at room temperature or at the physiological temperature of about 37° C. The redox couple is partitioned into separate reactive components A and B prior to use and then subsequently mixed at the time of use to generate the desired initiating species. Selection of the redox couple is governed by several criteria. For example, a desirable oxidizing agent is one that is sufficiently oxidizing in nature to oxidize the reducing agent, but not excessively oxidizing that it may prematurely react with other components with which it may be combined during storage. Oxidation of the resin with an inappropriate oxidizing agent could result in an unstable system that would prematurely polymerize and subsequently provide a limited shelf life.

Suitable oxidizing agents include peroxide compounds (i.e., peroxy compounds), including hydrogen peroxide as well as inorganic and organic peroxide compounds (e.g., “per” compounds or salts with peroxoanions). Examples of suitable oxidizing agents include, but are not limited to: peroxides such as benzoyl peroxide, phthaloyl peroxide, substituted benzoyl peroxides, acetyl peroxide, caproyl peroxide, lauroyl peroxide, cinnamoyl peroxide, acetyl benzoyl peroxide, methyl ethyl ketone peroxide, sodium peroxide, hydrogen peroxide, di-tert butyl peroxide, tetraline peroxide, urea peroxide, and cumene peroxide; hydroperoxides such as p-methane hydroperoxide, di-isopropyl-benzene hydroperoxide, tert-butyl hydroperoxide, methyl ethyl ketone hydroperoxide, and 1-hydroxy cyclohexyl hydroperoxide-1, ammonium persulfate, sodium perborate, sodium perchlorate, potassium persulfate, ozone, ozonides, 2-hydroxy-4-methoxy-benzophenone, 2 (2-hydroxy-5-methylphenyl)benzotriazol etc. Benzoyl peroxide is the preferred oxidizing agent. Other oxidizing agents include azo initiators, such as azoisobutyronitrile (AIBN) or 2,2-azobis (2-amidopropane)dihydrochloride.

These oxidizing agents may be used alone or in admixture with one another. One or more oxidizing agents may be present in an amount sufficient to provide initiation of the curing process. Preferably, this includes about 0.01 weight percent (wt-%) to about 4.0 wt-%, and more preferably about 0.05 wt-% to about 1.0 wt-%, based on the total weight of all components of the dental material.

Thus, suitable redox couples individually provide good shelf-life (for example, at least 2 months, preferably at least 4 months, and more preferably at least 6 months in an environment of 5-20° C.), and then, when combined together, generate the desired initiating species for curing or partially curing the curable admixture. The shelf life of the photoinitiator is dependent on the exposure to light. It is therefore preferred to store component A in an opaque container and/or in the dark. It is also preferred to formulate A such that oxidizers in the formulation do not react with the other components in the mixture and thereby reduce the shelf life.

In one particular embodiment, component A contains camphorquinone (CQ) and benzoyl peroxide (BPO). Preferably, the relative amounts (w/w) are between 5:1 and 1:5, more preferably between 2:1 and 1:2, and desirably about 1:1.

The light and chemical initiators are preferably dissolved in a liquid such as a PEG, PEG methacrylate, or a PEG dimethacrylate. Ethyl acetate, acetone, N-methyl-pyrrolidone, and/or N-vinyl pyrrolidone may also be added. The liquid primarily acts as a solvent for the initiator component and can be selected dependent on the viscosity desired for the mixture. Some of the solvents will also polymerize upon curing, and be incorporated into the polymer matrix (i.e., a reactive polymer). It may contain a reactive or non-reactive polymer that can be both a solvent and part of the shell polymer matrix. In addition to being a solvent, the liquid may also be used as a pore-generating agent (i.e., as the solvent evaporates, it leaves voids, or pores), or the liquid may have additional functionality.

When making component A, the order of mixing can be important to retain solubility and activity of the component. For example, in an embodiment containing CQ and BPO in a PEG and ethyl acetate mixture, the ethyl acetate should be mixed with the CQ and BPO before the PEG is added. It is also beneficial to obtain homogeneity in component A to obtain a good polymer cure.

In a second embodiment, Component A contains a chemical initiator but no photoinitiator.

Initiator Component B

In a first embodiment, component B comprises a light accelerator component (or co-catalyst) and a reducing agent. Exposure of the photoinitiator to light generates a triplet state which reacts with the light accelerator co-catalyst component to form an active species that initiates polymerization. Preferred co-catalysts are amines, and more particularly the aromatic amines. Examples of aromatic amine accelerators include: N-alkyl substituted alkylamino benzoates, such as 4-ethyl-dimethyl amino benzoate (4-EDMAB); N-alkyl benzylamines such as N,N-dimethylbenzylamine and N-isopropylbenzylamine; dibenzyl amine; 4-tolyldiethanolamine; and N-benzylethanolamine. Additionally, other suitable amine accelerators include N-alkyl-diethanolamines such as N-methyldiethanolamine; triethanolamine; and triethylamine. One particularly preferred aromatic amine is 4-EDMAB.

The reducing agent, which is also called a reducing component, is also in component B. A desirable reducing agent is one that is sufficiently reducing in nature to readily react with the preferred oxidizing agent, but not excessively reducing in nature such that it may reduce other components with which it may be combined during storage. Reduction of the resin with an inappropriate reducing agent could result in an unstable system that would prematurely polymerize and subsequently provide a limited shelf life.

A reducing agent has one or more functional groups for activation of the oxidizing agent. Preferably, such functional group(s) is selected from amines, mercaptans, or mixtures thereof. If more than one functional group is present, they may be part of the same compound or provided by different compounds. A preferred reducing agent is a tertiary aromatic amine (e.g., N,N-dimethyl-p-toluidine (DMPT) or N,N-bis(2-hydroxyethyl)-p-toluidine (DHEPT)). Examples of such tertiary amines are well known in the art and are disclosed, for example, in WO 97/35916 and U.S. Pat. No. 6,624,211. Another preferred reducing agent is a mercaptan, which can include aromatic and/or aliphatic groups, and optionally polymerizable groups. Preferred mercaptans have a molecular weight greater than about 200 as these mercaptans have less intense odor. Other reducing agents, such as some alcohols including methanol, ethanol, iso-propanol, and n-propanol, sulfinic acids, formic acid, ascorbic acid, hydrazines, and salts thereof, can also be used herein to initiate free radical polymerization.

If two or more reducing agents are used, they are preferably chosen such that at least one has a faster rate of activation than the other(s). That is, one causes a faster rate of initiation of the curing of the curable admixture than the other(s).

Electrochemical oxidation potentials of reducing agents and reduction potentials of oxidizing agents are useful tools for predicting the effectiveness of a suitable redox couple. For example, the reduction potential of the oxidant (i.e., oxidizing agent) benzoyl peroxide is approximately −0.16 volts vs. a saturated calomel electrode (SCE). Similarly, the oxidation potential (vs. SCE) for a series of amines has been previously established as follows: (e.g., N,N-dimethyl-p-toluidine ((DMPT), 0.61 volt), dihydroxyethyl-p-toluidine ((DHEPT), 0.76 volt), 4-t-butyl dimethylaniline ((t-BDMA), 0.77 volt), 4-dimethylaminophenethanol ((DMAPE), 0.78 volt), triethylamine ((TEA, 0.88 volt), 3-dimethylaminobenzoic acid ((3-DMAB) 0.93 volt), 4-dimethylaminobenzoic acid ((4-DMAB, 1.07 volts), ethyl p-dimethylaminobenzoate ((EDMAB), 1.07 volts), 2-ethylhexyl p-dimethylaminobenzoate ((EHDMAB), 1.09 volts) and 4-dimethylaminobenzoate ((DMABA), 1.15 volts). The ease of oxidation (and subsequent reactivity) increases as the magnitude of the oxidation decreases. Suitable amine reducing agents in combination with benzoyl peroxide generally include aromatic amines with reduction potentials less than about 1.00 volt vs. SCE. Less effective oxidants than benzoyl peroxide such as lauroyl peroxide (reduction potential=−0.60 volt) are poorer oxidizing agents and subsequently react more slowly with aromatic amine reducing agents. Suitable aromatic amines for lauroyl peroxide will generally include those having reduction potentials less than about 0.80 volt vs. SCE.

A preferred reducing agent is N,N-dimethyl-p-toluidine (DMT, also known as DMPT). When DMT is used, its percentage is preferably kept low to reduce heating of the sample that occurs during curing. It is preferred to keep the temperature below about 50° C. for the entire mixing process. In one particular exemplary embodiment, component B comprises 4-EDMAB and DMT in a ratio between 2:1 and 1:2.

In one embodiment, it is contemplated that a single agent (i.e., DMT) can be both the reducing agent and light accelerator of component B. This molecule must both have a suitable oxidation potential with the oxidizing agent and interact with the triplet state of the photoinitiator. In this embodiment, no other agent is required in component B.

It is contemplated that instead of an oxidizing agent in component A and reducing agent in component B, component A will contain a reducing agent and component B will contain the oxidizing agent. For this embodiment, the selection of the redox couple must be done with care so as not to provide a reducing agent that can act as an accelerator or otherwise react with the photoinitiator before the crosslinking is initiated by mixing the components.

In one embodiment, the present invention comprises an initiator system having only a chemical curing component. This initiator system is also divided into two parts, the first part (component A) comprising the chemical initiator and the second part (component B) comprises the chemical accelerator as discussed above.

Additional Initiators

Other initiators may also be added to the formulations of the present invention. Such initiators include additional photoinitiators or redox initiators. They also include thermal initiators, including peroxydicarbonate, persulfate (e.g., potassium persulfate or ammonium persulfate). Thermally activated initiators, alone or in combination with other type of initiators, are most useful where light can not reach (e.g., deep within the curable admixture). Additionally, multifunctional initiators may be used. These initiators may be added into component A or component B such that the initiator will not react with the other ingredients in component A or B before the crosslinking is initiated by mixing the components.

### IV. Optional Components in Crosslinkable Bone Substitute

The crosslinkable bone substitute of the present invention may contain the following optional components. These components may be mixed into the core particle, coated onto the core particle before the shell is applied, mixed with the shell material, or any combination thereof.

Excipients

One or more excipients may be incorporated into the compositions of the present invention. Non-limiting examples of such excipients include Ca(OH)₂, demineralized bone powder or particles, hydroxyapatite powder or particles, coral powder, resorbable and non-resorbable hydroxyapatite, calcium phosphate particles, α-tricalcium phosphate, octacalcium phosphate, calcium carbonate, and calcium sulfate. Preferably, such excipients can neutralize the acid generated during the degradation of a biodegradable polymer and maintain a physiological pH value suitable for bone formation. Preferably, such excipient is alkaline in nature so that it can neutralize the acid generated in the biodegradation process and help to maintain a physiological pH value. Steric acid is a preferred excipient. Steric acid is non-reactive and acts as a diluent. It can be used to increase hydrophobicity, reduce strength, and increase consistency of the polymer formulation. Ethyl acetate is another excipient that may be used to aid in the salvation and mixing as well as to obtain a viscosity useful for working with the polymerizable material.

Bone Promoting Agents

One or more substances that promote and/or induce bone formation may be incorporated into the compositions of the present invention. These agents may be incorporated into the core or the shell material. Agents incorporated in the core are preferably slowly released into the surrounding tissue as the core degrades over time.

The bone promoting agent can include, for example, proteins originating from various animals including humans, microorganisms and plants, as well as those produced by chemical synthesis and using genetic engineering techniques. Such agents include, but are not limited to: growth factors such as, bFGF (basic fibroblast growth factor), acidic fibroblast growth factor (aFGF) EGF (epidermal growth factor), PDGF (platelet-derived growth factor), IGF (insulin-like growth factor), the TGF-β superfamily (including TGF-βs, activins, inhibins, growth and differentiation factors (GDFs), and bone morphogenetic proteins (BMPs)), cytokines, such as various interferons, including interferon-α, -β, and γ, and interleukin-2 and -3; hormones, such as, insulin, growth hormone-releasing factor and calcitonin; non-peptide hormones; antibiotics; chemical agents such as chemical mimetics of growth factors or growth factor receptors, and gene and DNA constructs, including cDNA constructs and genomic constructs. In a preferred embodiment, the agents include those factors, proteinaceous or otherwise, which are found to play a role in the induction or conduction of growth of bone, ligaments, cartilage or other tissues associated with bone or joints, such as for example, BMP and bFGF. The present invention also encompasses the use of autologous or allogeneic cells encapsulated within the composition. The autologous cells may be those naturally occurring in the donor or cells that have been recombinantly modified to contain nucleic acid encoding desired protein products.

Non-limiting examples of suitable bone promoting materials include growth factors such as BMP (Sulzer Orthopedics), BMP-2 (Medtronic/Sofamor Danek), bFGF (Orquest/Anika Therapeutics), Epogen (Amgen), granulocyte colony-stimulating factor (G-CSF) (Amgen), Interleukin growth factor (IGF)-1 (Celtrix Pharmaceuticals), osteogenic protein (OP)-1 (Creative BioMolecules/Stryker Biotec), platelet-derived growth factor (PDGF) (Chiron), stem cell proliferation factor (SCPF) (University of Florida/Advanced Tissue Sciences), recombinant human interleukin (rhIL) (Genetics Institute), transforming growth factor beta (TGFβ) (Collagen Corporation/Zimmer Integra Life Sciences), and TGFβ-3 (OSI Pharmaceuticals).

The time required for bone formation within the pores of the bone substitute material may be reduced from several months to several weeks by the addition of a bone promoting agent to the bone substitute. Bone regenerating molecules, seeding cells, and/or tissue can be incorporated into the compositions. For example, bone morphogenic proteins such as those described in U.S. Pat. No. 5,011,691, the disclosure of which is incorporated herein by reference, can be used in these applications. For example bone morphogenic proteins such as those described in U.S. Pat. No. 5,011,691, the disclosure of which is incorporated herein by reference, can be used in these applications.

In one embodiment, the addition of a TGF-β superfamily member is particularly preferred. These proteins are expressed during bone and joint formation and have been implicated as endogenous regulators of skeletal development. They are also able to induce ectopic bone and cartilage formation and play a role in joint and cartilage development (Storm E E, Kingsley D M. Dev Biol. 1999 May 1; 209(1):11-27; Shimaoka et al., J Biomed Mater Res A. 200468(1):168-76; Lee et al., J. Periodontol. 2003 74(6):865-72). The BMP proteins that may be used include, but are not limited to BMP-1 or a protein from one of the three subfamilies. BMP-2 (and the recombinant form rhBMP2) and BMP-4 have 80% amino acid sequence homology. BMP-5, -6, and -7 have 78% % amino acid sequence homology. BMP-3 is in a subfamily of its own. Normal bone contains approximately 0.002 mg BMP/kg bone. For BMP addition to induce bone growth at an osseous defect, 3 to 3.5 mg BMP has been found to be sufficient, although this number may vary depending upon the size of the defect and the length of time it will take for the BMP to release. Additional carriers for the BMP may be added, and include, for example, inorganic salts such as a calcium phosphate or CaO4S. (Rengachary, S S., Neurosurg. Focus, 13(6), 2 (2002)). Particular GDFs useful in the present invention include, but are not limited to GDF-1; GDF-3 (also known asVgr-2); the subgroup of related factors: GDF-5, GDF-6, and GDF-7; GDF-8 and highly related GDF-11; GDF-9 and -9B; GDF-10; and GDF-15 (also known as prostate-derived factor and placental bone morphogenetic protein).

It is important for the bone promoting agent to remain active through the polymerization process. For example, many enzymes, cytokines, etc. are sensitive to the radiation used to cure polymers during photopolymerization and/or chemical polymerization. Therefore, the it may be advisable to protect the agents during the reaction. The method provided in Baroli et al., J. Pharmaceutical Sci. 92:6 1186-1195 (2003) can be used to protect sensitive molecules from light-induced polymerization. This method provides protection using a gelatin-based wet granulation. This technique may be used to protect the bone promoting agent incorporated into the polymer composition.

Porosity Forming Agents

One or more substances that promote pore formation may be incorporated into the composition of the present invention. Non-limiting examples of such substances include: particles of inorganic salts such as NaCl, CaCl₂, porous gelatin, carbohydrate (e.g., monosaccharide), oligosaccharide (e.g., lactose), polysaccharide (e.g., a polyglucoside such as dextrane), a gelatin derivative containing polymerizable side groups, porous polymeric particles, waxes, such as paraffin, bees wax, and carnauba wax, and wax-like substances, such as low melting or high melting low density polyethylene (LDPE), and petroleum jelly. Other useful materials include hydrophilic materials such as PEG, alginate, bone wax (fatty acid dimers), fatty acid esters such as mono-, di-, and tri-glycerides, cholesterol and cholesterol esters, and naphthalene. In addition, synthetic or biological polymeric materials such as proteins can be used.

The size or size distribution of the porosity forming agent particles used in the invention can vary according to the specific need. Preferably the particle size is less than about 5000 μm, more preferably between about 500 and about 5000 μm, even more preferably between about 25 and about 500 μm, and most desirably between about 100 and 250 μm.

Therapeutic Agents

One or more preventive or therapeutic active agents and salts or esters thereof may be incorporated into the compositions of the present invention, including but not limited to:

antipyretic analgesic anti-inflammatory agents, including non-steroidal anti-inflammatory drugs (NSAIDs) such as indomethacin, aspirin, diclofenac sodium, ketoprofen, ibuprofen, mefenamic acid, azulene, phenacetin, isopropylantipyrin, acetaminophen, benzydamine hydrochloride, phenylbutazone, flufenamic acid, mefenamic acid, sodium salicylate, choline salicylate, sasapyrine, clofezone or etodolac; and steroidal drugs such as dexamethasone, dexamethasone, sodium sulfate, hydrocortisone or prednisolone;

antibacterial and antifungal agents such as penicillin, ampicillin, amoxicillin, cephalexin, erythromycin ethylsuccinate, bacampicillin hydrochloride, minocycline hydrochloride, chloramphenicol, tetracycline, erythromycin, fluconazole, itraconazole, ketoconazole, miconazole, terbinafine; nlidixic acid, piromidic acid, pipemidic acid trihydrate, enoxacin, cinoxacin, ofloxacin, norfloxacin, ciprofloxacin hydrochloride, sulfamethoxazole or trimethoprim;

anti-viral agents such as trisodium phosphonoformate, didanosine, dideoxycytidine, azido-deoxythymidine, didehydro-deoxythymidine, adefovir dipivoxil, abacavir, amprenavir, delavirdine, efavirenz, indinavir, lamivudine, nelfinavir, nevirapine, ritonavir, saquinavir or stavudine;

high potency analgesics such as codeine, dihydrocodeine, hydrocodone, morphine, dilandid, demoral, fentanyl, pentazocine, oxycodone, pentazocine or propoxyphene; and

salicylates which can be used to treat heart conditions or as an anti-inflammatory.

The agents can be incorporated in the composition of the invention directly, or can be incorporated in microparticles which are then incorporated in the composition. Incorporating the agents in microparticles can be advantageous for those agents which are reactive with one or more of the components of the composition.

The method described in Baroli et al., J. Pharmaceutical Sci. 92:6 1186-1195 (2003) can be used to protect sensitive therapeutic agents from light-induced polymerization when incorporated in the polymer composition.

Diagnostic Agents

One or more diagnostic agents may be incorporated into the compositions of the present invention. Diagnostic/imaging agents can be used which allow one to monitor bone repair following implantation of the compositions in a patient. Suitable agents include commercially available agents used in positron emission tomography (PET), computer assisted tomography (CAT), single photon emission computerized tomography, X-ray, fluoroscopy, and magnetic resonance imaging (MRI).

Examples of suitable agents useful in MRI include the gadolinium chelates currently available, such as diethylene triamine pentaacetic acid (DTPA) and gadopentotate dimeglumine, as well as iron, magnesium, manganese, copper and chromium gadolinium chelates.

Examples of suitable agents useful for CAT and X-rays include iodine based materials, such as ionic monomers typified by diatrizoate and iothalamate, non-ionic monomers such as iopamidol, isohexyl, and ioversol, non-ionic dimers, such as iotrol and iodixanol, and ionic dimers, for example, ioxagalte.

These agents can be detected using standard techniques available in the art and commercially available equipment.

Stabilizing Agents

Agents may be added to stabilize one or more of the compounds. The stabilizer may be a compound designed to remove free radicals and prevent premature polymerization. One stabilizer, methylhydroquinone, is also an antioxidant and prevents polymerization of the acrylic monomers. It is contemplated that additional initiator may be added to the mixture when the polymer contains a stabilizing agent to counter the effect of the stabilizing agent as well as polymerize the compound.

### V. Properties of the Crosslinkable Bone Substitute

Strength

The strength required for the bone substitute is dependent upon the application; some applications require an implant that is load bearing or has significant torsional strength so that the patient can use the area between the time of implantation and when bone growth has replaced the implant material. Other applications do not require the implant to have much strength, for example, the implant used to prevent jaw bone loss. It is preferred that the strength of the crosslinked composite be from about 5 to 300 N/m²; more preferably from about 20 to 200 N/m²; and most desirably from about 50 to 200 N/m².

Porosity

High porosity is an important characteristic of the present invention. The bone substitute is porous to allow bone growth within the scaffold of the bone substitute. This porosity includes the interstitial region between the particles when packed into an implant. Therefore, the shell material must not encompass all of this region.

Biodegradation/Bioresorption Duration

The time needed for biodegradation/bioresorption of the crosslinked composite can be varied widely, from days to years. The suitable biodegradation/bioresorption duration depends on a number of factors such as the speed of osteointegration, whether the compositions are functional and/or load-bearing, and/or the desirable rate of drug release. For example, osteointegration in an elderly woman is typically much slower than that in a 20 year old man. When osteointegration is slow, a composition having a long biodegradation/bioresorption time should be used. An immediately functional dental implant is load-bearing and must remain strong during osteointegration, so a long biodegradation/bioresorption composition is more suitable for application around such dental implant.

The degradation time is a function of the hydrophobicity/hydrophilicity of the components. A more hydrophobic polymer has a longer degradation time. The degradation time is also a function of geometrical shape, thickness, etc.

The biodegradation of the material is also important for the delivery of therapeutic agents into the tissue of blood surrounding the implant. This slow release of agent provides a supply of the therapeutic agent over an extended period of time as the bone grows into to porous material.

Micro-Movement

The amount of micro-movement the implant will be subject to can be an important consideration. It is contemplated that in one embodiment, the bone substituted is formulated to have very little movement.

Viscosity

The viscosity of the crosslinkable bone substitute can vary widely. It depends on a number of factors such as the molecular weight of the ingredients in the crosslinkable bone substitute, and the temperature of the crosslinkable bone substitute. Typically, when the temperature is low, the crosslinkable bone substitute is more viscous; and, when the average molecular weight of the ingredients is high, it becomes more viscous. Different applications of the crosslinkable bone substitute also require different viscosities. For example, to be injectable, the admixture must be a free flowing liquid and, in other applications, it must be a moldable paste-like putty.

Hydrophobicity/Hydrophilicity

The hydrophobicity/hydrophilicity of the crosslinkable bone substitute should be carefully controlled. Preferably, the crosslinkable bone substitute is sufficiently hydrophilic that cells adhere well to them. The hydrophobicity/hydrophilicity depends on a number of factors such as the hydrophobicity/hydrophilicity of the crosslinkable bone substitute. For example, when the bone substitute is a PMMA/PHEMA based polymer particle, the ratio of PMMA (less hydrophilic) and PHEMA (more hydrophilic) affects the hydrophobicity/hydrophilicity.

### VI. Preferred Embodiments of the Method of Crosslinking the Bone Substitute

The core and shell bone substitute comprising a plurality of coated polymer beads is crosslinked to form the crosslinked composite. More specifically, the crosslinkable reactive groups comprising the outer polymeric material of the bone substitute crosslink with each other, forming the crosslinked composite.

When the core and shell material is formed, the two materials are mixed together to physically coat the shell material on the core particles. The amount of shell material required is dependent upon the size of the particles and the thickness of the shell to be formed, however, in each embodiment, the core polymer will comprise the majority of the bone substitute compared to the polymer coat by weight.

The coverage and thickness of the shell over the core particle can be adjusted by varying the concentration of the shell monomer mixed with the core particles. By reducing the relative percent of the shell material, more core surface area will be exposed. It is important to provide enough coverage of the shell to provide strong and stable linkages between the particles, but substantial amounts of the core may remain without a shell layer separating it from the surrounding environment.

In one embodiment, the shell completely surrounds and coats the core particles. In another embodiment, the shell only partially covers the core particles, such that a Ca(OH)₂ surface coating on the core particles is partially exposed to the environment, which, after application as an implant, will interact with the blood and induce bone growth as described in U.S. Pat. No. 4,728,570.

For example the core/shell weight ratio can be 60/40, 70/30, 80/20, 90/10, 95/5, or higher. Preferably, the ratio is at least 80/20. When 750 μm Bioplant® HTR® particles are used as the core material, less than 1.0% shell material will provide a 1 μm thick layer on the particle surface if it is evenly coated. (the Bioplant® HTR® surface area is approx. 1.77×10² cm²; with a bead density for PMMA (d=1.2 cm³/g) of 66.7 cm³/g of beads, a 1 μm thick layer of polymer (PEG at d=1.1 g/cm³) requires 7.3 mg/g of HTR). However, a large range of surface layer thicknesses (or the thickness of a layer only partially covering the bead) will be appropriate in the present invention.

In one preferred embodiment of the present invention, Bioplant® HTR® is improved upon by adding a polymeric shell. In the present invention, the shell of Bioplant® HTR® comprises an agent having at least one crosslinkable reactive group and optionally at least one spacer moiety. Consequently, the improved Bioplant® HTR® comprises microporous particles of calcified (Ca(OH)₂/calcium-carbonate) copolymer of PMMA and a PHEMA, PEG, or modified PHEMA material.

The shell can be formed by mixing an amount of the shell monomer material with the core particles until the particles are evenly coated. This can be done in the presence or absence of a solvent material.

The crosslinking can take place in situ, ex vivo or in vivo, and is done using an initiator.

The curable admixture is crosslinked through the use of initiator component A and B and, when a photochemical initiator is used, light to form the cured composite. The components are mixed thoroughly with the polymer or prepolymer(s). A ball mixer may be used to improve the consistency of mixing.

It is important to keep component A separated from component B before initiating polymerization so that the materials within the two components do not react or cure before the polymerization reaction is started. It is similarly important to keep component A separated from the polymers or polymerizable material before use since the photochemical initiator can initiate at least some polymerization without the presence of the accelerator.

The concentration of the initiator(s) used is dependent on a number of factors. Non-limiting examples of such factors include the type of the initiator, whether the initiator is used alone or in combination with other initiators, the desirable rate of curing, and how the material is applied. The concentration of each initiator is between about 0.05% (w/w) to about 5% (w/w) of the crosslinkable prepolymer. Preferably, the concentration is less than 1% (w/w) of the crosslinkable prepolymer, more preferably between 0.05 and 0.1% (w/w). In one embodiment, 20 μl of component A (0.5/ml total initiators) and 20 μl of component B (0.4 g/ml total initiators) are added per gram of polymer. In another embodiment, 40 μl of each component is added per gram of polymer to effect a stronger polymer.

It is preferred to utilize a particular sequence of adding the initiator components A and B, since mixing in any other order could drastically reduce the amount or homogeneity of the polymerization reaction. In one illustrative embodiment, component A is mixed with the polymer or prepolymer until evenly dispersed. Next, component B is mixed into the composition. If the mixing of component B was rapid, the mixture should be allowed to stand for about 10-30 seconds (with optional occasional mixing). The viscosity of the mixture should noticeably increase. At this point, it is possible to transfer into a mold or inject into a space in which the polymerization should occur. Light is then directed onto the sample for 0.5, 1, 2, 3, or more minutes to complete curing. Preferably, the polymer will cure in one minute or less. The light may, for example, be a UV, white, or blue light. A dental blue light (e.g., a Demitron or a 3M light) may be used. Most of the photo-initiated curing should occur within one minute, however, longer exposure to the light is also acceptable.

Samples of up to 1.5 cm have been cured in this manner. It is possible to cure thicker samples that are less opaque or where the chemical curing provides substantially more of the cure in the sample section farther from the light source. The size and shape of the sample is a factor in the curing of the polymer; thicker samples will take longer to cure. Additionally, larger samples may not receive the same exposure to the light source across the sample surface due to the size of the source and variations in light intensity. Since many light sources have a Gaussian profile, it may be advisable to move either the sample or the light source across the sample surface during curing to effect an evenly cured composite.

In the embodiments of the present invention where only chemical curing is used, components A and B will contain the redox component but not the photocuring agents. In one such preferred embodiment, in which component A contains benzoyl peroxide and component B contains DMT, these can be combined to initiate curing in a molar ratio of approximately 1:1. The same initiator concentration as used for combined light and chemical curing may be used for chemical-only curing, and is preferably below 1%.

In one embodiment, the core bead structure, the crosslinkable monomer or polymer, and initiator B are combined prior to use. This mixture is mixed with initiator component A when the composite material is needed, forming a simple two-phase system. The material is then packed in the bone cavity or other area, and light is directed onto the mixture to initiate polymerization.

The crosslinkable bone substitute is subjected to electromagnetic radiation from a radiation source for a period sufficient to crosslink the bone substitute and form a crosslinked composite. Preferably, the crosslinkable bone substitute is applied in layer(s) of 1-10 mm, more preferably about 3-5 mm, and subjected to an electromagnetic radiation for about 30 to 300 seconds, preferably for about 50 to 100 seconds, and more preferably for about 60 seconds.

Typically, a minimum of 0.01 mW/cm² intensity is needed to induce polymerization. Maximum light intensity can range from 1 to 1000 mW/cm², depending upon the wavelength of radiation. Tissues can be exposed to higher light intensities, for example, to longer wavelength visible light, which causes less tissue/cell damage than shortwave UV light. In dental applications, blue light is used at intensities of 100 to 400 mW/cm² clinically. When UV light is used in situ, it is preferred that the light intensity is kept below 20 mW/cm².

In another embodiment, when a thermally activated initiator is used (alone or in combination with other type(s) of initiator(s)), the crosslinkable bone substitute is subjected to a temperature suitable for activating the thermally activated initiators, preferably at a temperature from about 20 to 80° C., more preferably from about 30 to 60° C. Heat required to activate the thermal activator can be generated by various known means, including but not limited to infrared, water bath, oil bath, microwave, ultrasound, or mechanical means. For example, one can place the bone substitute in a crucible heated by a hot water bath.

In yet another embodiment, when a redox initiator system is used (alone or in combination with other type(s) of initiator(s)), the oxidizing agent of the redox initiator system is kept apart from the reducing agent of the redox initiator system until immediately before the curing process. For example, the oxidizing agent is mixed with some crosslinkable bone substitute in one container and the reducing agent is also mixed with some crosslinkable bone substitute in another container. The contents of the two containers are mixed with each other at which point substantial crosslinking is initiated.

In a most preferred embodiment, in order to shorten the duration of the radiation exposure and/or increase the thickness of the radiation crosslinkable layer, a redox initiator system is used in combination with a photoinitiator and/or thermal initiator. For example, the redox initiator system is activated first to partially crosslink the crosslinkable bone substitute. Such partially crosslinked bone substitute is then subjected to radiation and the photoinitiator and/or thermal initiator is activated to further crosslink the partially crosslinked admixture.

The bone substitute material is used to replace bone and other hard tissue. In addition, the bone substitute material can be used to replace soft tissue. The core material, Bioplant® HTR®, has been shown to slowly resorb in soft tissue as well as hard tissue. Particularly in the dental arts, aesthetics are an important consideration during the bone replacement. Soft tissue may be modified in order to make the gums and any other tissue surrounding the implant area more attractive by adding the bone substitute material in the soft tissue surrounding the implant to plump it up.

As used herein: “Electromagnetic radiation” refers to energy waves of the electromagnetic spectrum including, but not limited to, X-ray, ultraviolet, visible, infrared, far infrared, microwave, radio-frequency, sound and ultrasound waves. “Ultraviolet light” refers to energy waves having a wavelength of at least approximately 1.0×10⁻⁶ cm but less than 4.0×10⁻⁵ cm. “Visible light” refers to energy waves having a wavelength of at least approximately 4.0×10⁻⁵ cm to about 7.0×10⁻⁵ cm. “Blue light” refers to energy waves having a wavelength of at least approximately 4.2×10⁻⁵ cm but less than 4.9×10⁻⁵ cm. “Radiation source” as used herein refers to a source of electromagnetic radiation. Examples include, but are not limited to, lamps, the sun, blue lamps, and ultraviolet lamps.

### VII. Applications of the Crosslinked Bone Substitute of the Invention

Dental

The crosslinkable bone substitute and crosslinked composite of the present invention can be used to fill extraction sockets; prevent or repair bone loss due to tooth extraction; repair jaw bone fractures; fill bone voids due to disease and trauma; stabilize an implant placed into an extraction socket and one placed into an edentulous jawbone to provide immediate function (e.g., chewing); provide ridge (of bone) augmentation; repair periodontal bone lesions; and provide esthetic gingiva reshaping and plumping.

For the foregoing applications, the crosslinkable bone substitute can be crosslinked by exposure to electromagnetic radiation and/or heat and applied using standard dental or surgical techniques. The crosslinkable bone substitute may be applied to the site where bone growth is desired and crosslinked to form the crosslinked composite. The crosslinkable bone substitute may also be pre-cast into a desired shape and size (e.g., rods, pins, screws, and plates) and crosslinked to form the crosslinked composite.

Orthopedic

The crosslinkable bone substitute and crosslinked composite of the present invention can be used to repair bone fractures, fix vertebrae together, repair large bone loss (e.g., due to disease) and provide immediate function and support for load-bearing bones; to aid in esthetics (e.g., chin, cheek, etc.)

The crosslinkable bone substitute can be applied for the above purposes using standard orthopedic or surgical techniques; e.g., it can be applied to a site where bone generation is desired and crosslinked to form the crosslinked composite. For example, the admixture can be applied into the intervertebral space. The crosslinkable bone substitute may also be pre-cast into a desired shape and size (e.g., rods, pins, screws, plates, and prosthetic devices such as for the skull, chin, and cheek) and crosslinked to form the crosslinked composite.

Drug Delivery

The crosslinkable bone substitute and crosslinked composite of the present invention may be used to deliver therapeutic or diagnostic agents in vivo. Examples of drugs or agents which can be incorporated into such compositions include proteins, carbohydrates, nucleic acids, and inorganic and organic biologically active molecules. Specific examples include enzymes, antibiotics, antineoplastic agents, local anesthetics, hormones, angiogenic agents, antiangiogenic agents, antibodies, neurotransmitters, psychoactive drugs, drugs affecting reproductive organs, and oligonucleotides such as antisense oligonucleotides.

### EXAMPLES

The following examples are intended to illustrate more specifically the embodiments of the invention. It will be understood that, while the invention as described therein is a specific embodiment, the description and the example are intended to illustrate and not limit the scope of the invention. Other aspects, advantages, and modifications within the scope of the invention will be apparent to those skilled in the art to which the invention pertains. Formulations made with Bioplant® HTR® core polymer obtained from Bioplant®. Other polymers, initiators, and monomers were obtained from Aldrich™ except for DMEAMA obtained from Pfaultz & Baur.

### Example 1

### Bioplant® HTR®+HEMA

Bioplant® HTR® core was mixed with the HEMA monomer(s) for 5-7 minutes prior to addition of initiator solutions. This mixture was left for a time (set time) before adding initiators; this allows for excess monomer to settle out of the Bioplant® HTR® mixture.

Two drops of initiator composition A containing CQ/BPO in ethyl acetate (5:95) was first mixed. Two drops of initiator composition B was then incorporated into Bioplant® HTR®/monomer mixture (3-5 min), where composition B contains DMPT/EDMAB in PEG-DM (5:95). The mixture was then transferred to a mold and cured for 1 minute, unless otherwise specified. Light was provided by a Flashlite 1001t™ LED Dental Curing Light.

Bioplant® HTR® (0.2963, 0.2885, 0.2938, 0.2883, and 0.3034 g) was mixed with HEMA monomer (0.0798, 0.0768, 0.0733, 0.0761, and 0.0871 g) to provide coated core particles having 81-93% Bioplant® HTR®. The percent Bioplant® HTR® is determined after the excess monomer was allowed to settle out of the polymer mixture. The set time ranged from 0-60 seconds, with little difference noted between the trial runs. Each of these samples provided a hard polymer material.

### Example 2

### Bioplant® HTR®+PEG-DM

The procedure described in Example 1 was used for samples containing PEG-DM and HEMA monomers. In this experiment, Bioplant® HTR® (0.2725, 0.2459, 0.2542, 0.2558, and 0.2455 g) was mixed with PEG-DM (2% wt)/HEMA monomer (0.0699, 0.0664, 0.0769, 0.0714, and 0.0768 g) to provide coated core particles having 77-81% Bioplant® HTR®. The set time ranged from 0-60 seconds, with little difference noted between the trial runs. The two initiators (2 drops CQ in EA and 2 drops EDMAB in PEG-DM) were then incorporated into the mixture and mixed will (for 3-5 minutes). The mixture was then transferred to either a clean glass and cured to provide a hard polymeric substrate for each of the samples.

### Example 3

A number of different monomers (PEG-DM, HEMA, and 10% EG-DM in HEMA) were mixed with Bioplant® HTR® and the initiators were added as described in Example 1.

Initiator composition A containing CQ/BPO in ethyl acetate (5:5:90). Initiator composition B contained DMPT/EDMAB in PEG-DM (5:5:90). The mixtures were transferred to 5 mm×10 mm Teflon™ molds and cured for 1 minute with a Flashlite 1001t™ LED Dental Curing Light to form a hard material.

The first sample was made by adding 15% PEG-DMA to 85% Bioplant® HTR®.

The second polymer was made by adding 20% HEMA to 80% Bioplant® HTR®.

The third polymer was made by adding 20% of a mixture of 10% PEG-DMA and 90% HEMA to 80% Bioplant® HTR®.

### Example 4

The following table provides the various shell materials and weights used according to the process described in Example 1, with the monomer evenly coating the core beads with the exception of the MMA sample where the monomer appeared to dry up when contacted with the core polymer. The set time is 0 min, and samples were analyzed without removal to a mold.

| | | Percentage | Observations |
|---|---|---|---|
| HTR ® (g) | Monomers(s) (g) | HTR ® | After Curing |
| 0.3045 | PEG-DM 330 | 66% | hardened |
| | 0.1562 | | |
| 0.2540 | PEG-DM 330 | 81% | hardened |
| | 0.0582 | | |
| 0.2420 | MMA | 66% | 2 min. hard in few |
| | 0.121 | | places, falls apart |
| 0.2653 | HEMA | 77% | hardened |
| | 0.0810 | | |
| 0.2545 | PEG-MA | 78% | Hard in few |
| | 0.0712 | | places, falls apart |
| 0.2988 | HEMA:PEG-DM | 78% | hardened |
| | 0.0830 | | |
| | (25:75) | | |
| 0.2540 | HEMA:PEG-DM | 78% | hardened before |
| | 0.0724 | | cure |
| | (50:50) | | |
| 0.2163 | HEMA:PEG-DM | 75% | hardened |
| | 0.0693 | | |
| | (75:25) | | |

### Example 5

Bioplant® HTR® particles were combined with HEMA or PEG-DM to create particles with HEMA or PEG-DM shells while using an initiator system having only light-curing properties.

Two drops of initiator composition A containing CQ in ethyl acetate (5:95) was used. Two drops of initiator composition B was also used, where composition B contains EDMAB in PEG-DM (5:95).

| | coating | | Observations After |
|---|---|---|---|
| HTR ® (g) | (g) | Percent HTR ® | Curing |
| 0.2483 | 0.0521 | 82% | Hardened in few places; |
| | HEMA | | sample falls apart |
| 0.3127 | 0.0813 g | 79% | Hardened in few places; |
| | PEG-DM | | sample falls apart |

### Example 6

Bioplant® HTR® particles were combined with HEMA or PEG-DM to create particles with HEMA or PEG-DM shells while using an initiator system having only chemical, or redox curing properties.

Two drops of initiator composition A containing BPO in ethyl acetate (5:95) was used. Two drops of initiator composition B containing DMPT in PEG-DM (5:95) was used.

| Bioplant ® | | Percent | |
|---|---|---|---|
| HTR ® | coating | Bioplant ® | Observations After |
| (g) | (g) | HTR ® | Curing |
| 0.2806 | 0.0712 g | 80% | very hard with a yellow |
| | HEMA | | tint |
| 0.2575 | 0.0766 g | 77% | Hardened in few places; |
| | PEG-DM | | sample falls apart |

### Example 7

Bioplant® HTR® (0.25 g) obtained from Bioplant® can be mixed with monomeric HEMA (0.80 g) and two drops of initiator B (DMPT/EDMAB in PEG-DM (5:5:90) for 5 minute. This material can then be stored, packaged, or shipped. When ready for use, initiator A (CQ/BPO in ethyl acetate (5:5:90)) can then be mixed into this material until homogeneous (3-5 min.) The bone substitute is placed in a mold and cured for 1 minute using a Flashlite 1001t™ LED Dental Curing Light.

### Example 8

### Mechanical Testing

The surface morphology of the polymeric beads having a crosslinkable shell formed in Example 3 underwent mechanical testing and visualization using SEM. Three formulations were tested:

Formulation 1 HTR: PEG-DM (82/18).

Formulation 2 HTR:HEMA (80/20), and

Formulation 3 HTR:PEG-DM/HEMA (10% w/w).

The mechanical properties were determined using uniaxial compression at low uniform rates of straining or loading with standard shapes. Averages and standard deviations (SI units) were used. The properties of interest include the morphological features pore size and porosity and the mechanical properties: modulus of elasticity, proportional limit, compressive yield strain, compressive yield strength, and crushing load. An unconstrained uniaxial compression test at room temperature with a 500 N load cell was used. Strain was calculated from crosshead displacement. Stress was calculated from the load and cross-sectional area.

Right cylinders approximately 5 mm in diameter and 10 mm in height were used. The diameter of each sample was measured by a Mitutoyo digital caliper to the nearest 0.01 mm at several points along its length. A concentric semi-circular mold (ID 5 mm, OD 50 mm) was made to precisely mount the specimen at the center of the bottom anvil. All specimens were tested at 24° C. and ambient humidity. The test was run at 1.0 mm/min; for relatively ductile, the speed was increased to 6 mm/min after the yield point was reached. Loads and corresponding compressive strain were measured as well as the maximum load carried by the sample. Tests were stopped when the samples were crushed to failure.

### Mechanical Properties

The sample of Formulation 1 was tackier than either the sample containing Formulations 2 or 3. Upon crushing, none of the specimens of Formulation 1 completely broke; rather they were squeezed and deformed. Formulations 2 and 3 were harder, but were also more brittle, all specimens of which were crushed and fragmented under sufficient load.

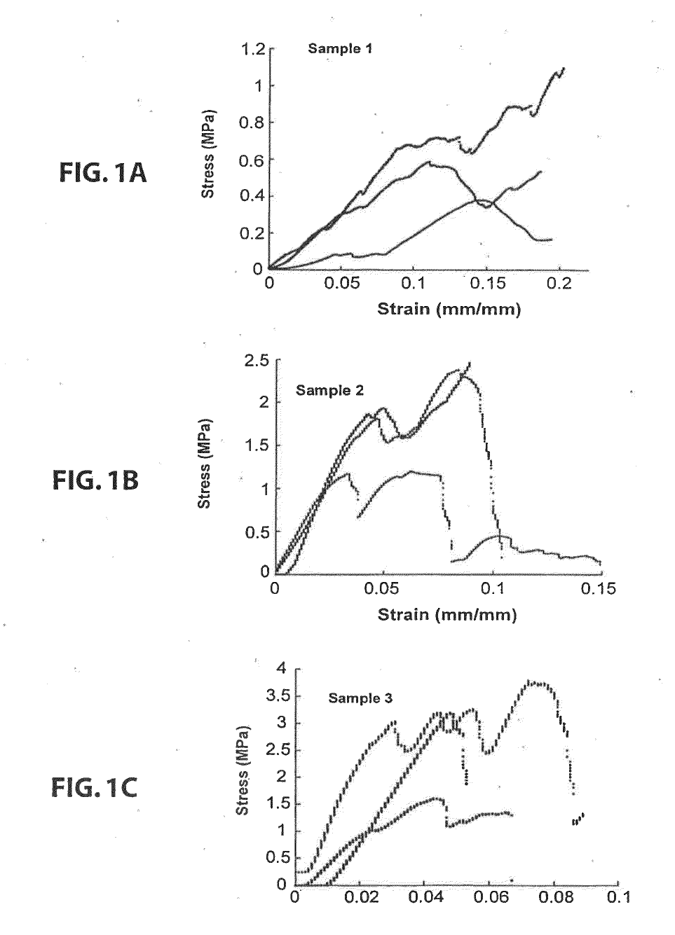

The ends of several specimens were not parallel to each other, which compromised the accuracy of the mechanical testing. Compressive stress-strain diagrams are shown in FIGS. 1A, 1B, and 1C. The initial “toe” region, where the stress changes gradually and non-linearly with the strain, does not represent the property of the material. It is due to take up of slack, alignment or seating of the specimen, and compression of the pointed ends in the few samples where the ends are further off parallel. Therefore, the strain, modulus, and offset limit were all calculated after the toe region was compensated, per guidance of ASTM standard. (ASTM standard D695-02a. “Standard Test Method for Compressive Properties of Rigid Plastics,” ASTM international, Aug. 10, 2002).

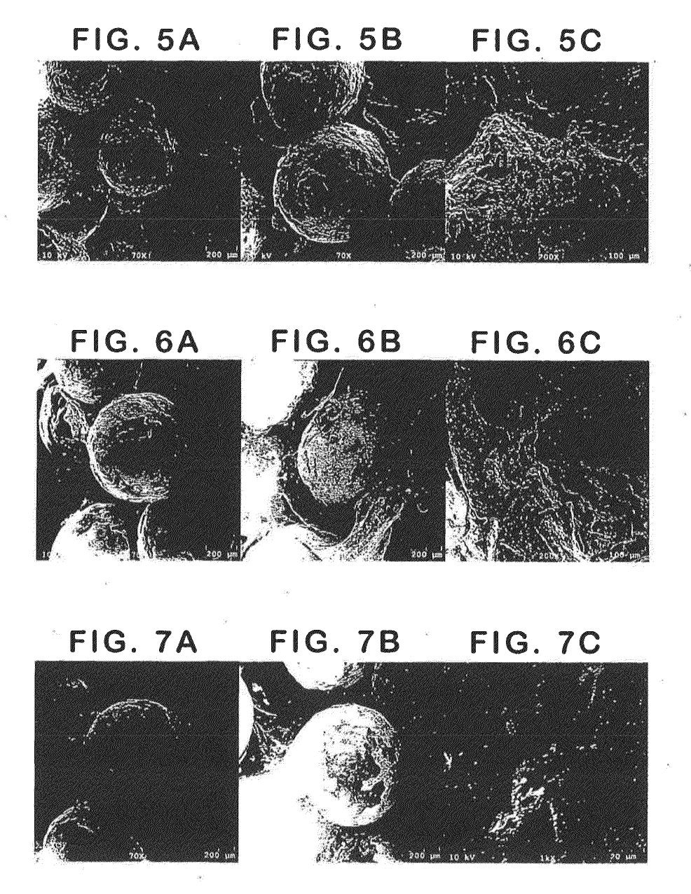

Formulation 1 showed steps in the stress-strain curve, most likely due to the crushing of layers of hollow or porous spheres, which was confirmed in the SEM observation of the crushed specimens (FIG. 5). The defects eventually accumulated sufficiently to cause the complete failure of the specimens. The specimens were relatively soft and tacky, thus instead of being crushed into fragments, the sample was deformed or substantially shortened Formulations 2 and 3 were stiffer than Formulation 1, as indicated in the table. The strongest sample in terms of crushing load is Formulation 3, however, it also has the lowest yield strain, which means it can't be deformed as much as the other two before being crushed. It is to be noted that the values were for the cross-sectional area of about 18 mm². Assuming a dental implant will be of 1 cm², the crushing load will be approximately 5 times larger.

Compressive mechanical properties of coated polymers. Data expressed as mean ± SD

| | Elastic | | | Comp. yield | |
|---|---|---|---|---|---|
| | modulus | Proportional | Comp. yield | strength | Crushing |
| Sample | (MPa) | limit (MPa) | strain (%) | (N) | load (N) |
| HTR:PEG-DM | 7.67 ± 2.39 | 0.520 ± 0.163 | 11.7 ± 2.42 | 9.69 ± 2.52 | 12.4 ± 6.63 |
| (82/18) | | | | | |
| HTR:HEMA | 53.4 ± 10.9 | 1.17 ± 0.328 | 4.10 ± 0.681 | 29.8 ± 7.19 | 37.2 ± 12.8 |
| (80/20) | | | | | |
| HTR:EG-DM/HEMA | 101 ± 45.3 | 1.98 ± 1.09 | 4.07 ± 0.895 | 47.2 ± 16.5 | 52.6 ± 21.1 |
| (10% w/w) (80/20) | | | | | |

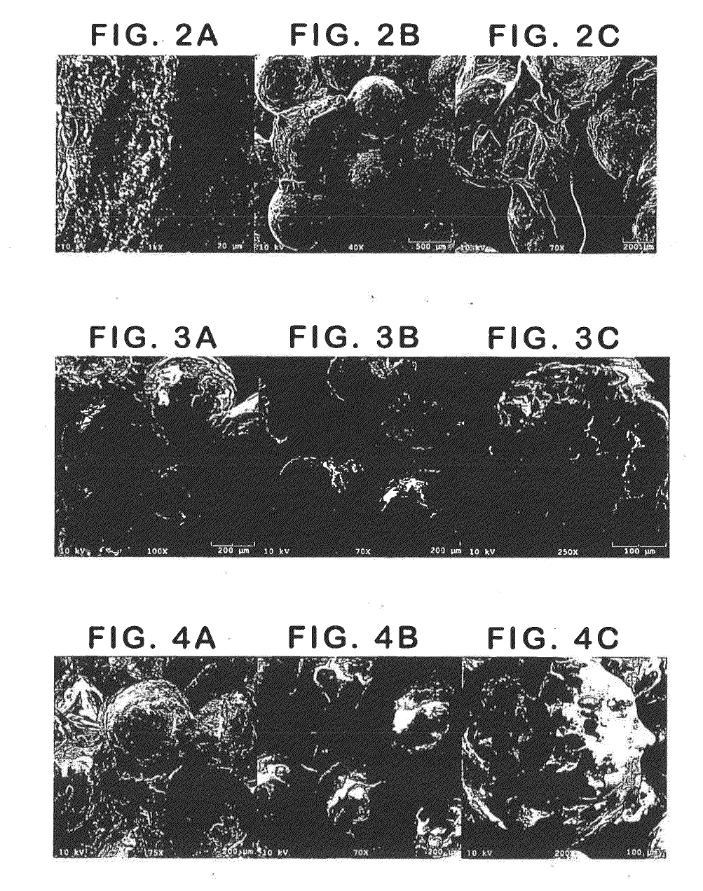

Images were viewed using a Hitachi S-800 SEM (10 kV, 3-5 nm spot size). Samples after compression test were sputtered with gold before SEM observation to enhance image quality. Pristine samples are shown in FIGS. 2-4, and crushed ones are shown in FIGS. 5-7.

In FIGS. 2 (2A, 2B, and 2C), all Formulation 1 specimens appeared to be made of fused hollow spheres. A few spheres seemed to have ‘craters’ as if erupted by a sudden increase in internal pressured, however, higher magnification images (1000× and 2000×) revealed that all ‘craters’ were covered with a skin. In FIGS. 3 (3A, 3B, and 3C), the samples made with Formulation 2 are seen to have pores of ˜250 μm, apparently formed when the individual spheres erupted during manufacturing process. Approximate porosity is around 6-8% from image analysis. Formulation 3 (FIG. 4, including 4A, 4B, and 4C) also displayed ruptured-bead morphology. The rupture appeared more violent than those in Formulation 2 and the edges were more jagged. The average pore size was about 150-200 μm. Milder mixing or molding conditions or a new batch of Bioplant® HTR® should reduce or alleviate the ruptures.

The surface morphology after compression testing is shown in FIGS. 5-7. Being relatively soft and tacky, the spheres of Formulation 1 (FIG. 5, including 5A, 5B, and 5C) were not broken but rather flattened. No pores were observed on surface even at 1000× magnification. Interestingly, the pores for Formulation 2 (FIG. 6, including 6A, 6B, and 6C) disappeared after the specimens were crushed; minor cracks were visible on the surfaces. Formulation 2 (FIG. 7, including 7A, 7B, and 7C) has visible cracks on some of the bead surfaces. The sample almost appeared intact other than the cracks.

The elastic modulus and the crushing load increase from the sample containing PEG-DM to HEMA, and then to EG-DM+HEMA, while the strain at break decreases. When the samples failed under compressive load, the PEG-DM-containing sample (Formulation 1) did not fragment, showing superior strength under compressive load. Samples of Formulation 2 and 3 fragmented at a crushing load.

What is claimed:

1. A crosslinkable bone substitute material comprising micron sized particles, each particle having a core comprising one or more first biologically-compatible material(s), and
a shell generally surrounding the core, the shell comprising one or more second biologically-compatible polymer or polymerizable material(s) having at least one crosslinkable reactive group;
wherein the bone substitute material has interstices between the particles forming pores into which bone tissue can grow; and
wherein the shell forms a crosslinked polymer upon curing by light and/or redox chemistry.

2. The bone substitute of claim 1, wherein the core comprises a polymeric alloplast.

3. The bone substitute of claim 1, wherein the core comprises polymethylmethacrylate and polymeric hydroxyethylmethacrylate.

4. The bone substitute of claim 3, wherein the core comprises calcium hydroxide distributed on the outer surfaces of the core particles.

5. The bone substitute of claim 3, wherein the core comprises intra-particle pores and extra-particle pores into which bone tissue can grow.

6. The bone substitute of claim 1, wherein the core comprises a ceramic or ceramic/polymer hybrid.

7. The bone substitute of claim 6, wherein the core comprises a hydroxyapatite, tricalcium phosphate, or mixture thereof.

8. The bone substitute of claim 1, wherein the shell comprises a hydrophilic polymer.

9. The bone substitute of claim 1, wherein the shell comprises a polymer or prepolymer comprising a vinyl group.

10. The bone substitute of claim 9, wherein the shell comprises hydroxyethylmethacrylate, poly(ethylene glycol)diacrylate, poly hydroxyethylmethacrylate, or a combination thereof.

11. The bone substitute of claim 10, wherein the shell comprises hydroxyethylmethacrylate.

12. The bone substitute of claim 10, wherein the shell comprises poly(ethylene glycol)diacrylate.

13. The bone substitute of claim 1, wherein the shell is crosslinked using a photoinitiator blue dental light or a UV light.

14. The bone substitute of claim 1, further comprising a bone or soft tissue growth factor or a therapeutic agent.

15. The bone substitute of claim 14, wherein the growth factor or therapeutic agent is protected by gelatin-based wet granulation.

16. The bone substitute of claim 14, wherein the growth factor is a steroid or an antibiotic.

17. The bone substitute of claim 14, wherein the therapeutic agent is a bone morphogenic protein.

18. The bone substitute of claim 14, wherein the growth factor or therapeutic agent is released slowly from the bone substitute.

19. A crosslinked bone substitute comprising a matrix of micron sized particles, each particle having a core comprising one or more first biologically-compatible material(s) and a shell generally surrounding the core, the shell comprising one or more second biologically-compatible polymeric material(s);
wherein each particle shell has at least one crosslinked moiety electrostatically or chemically bound to a crosslinked moiety of a different particle shell;
wherein the bone substitute has interstices between the particles forming pores into which bone tissue can grow.

20. The bone substitute of claim 19, wherein the core comprises a polymeric alloplast.

21. The bone substitute of claim 19, wherein the core comprises polymethylmethacrylate and polymeric hydroxyethylmethacrylate.

22. The bone substitute of claim 21, wherein the core comprises calcium hydroxide distributed on the outer surfaces of the core particles.

23. The bone substitute of claim 19, wherein the core comprises a ceramic or ceramic/polymer hybrid.

24. The bone substitute of claim 19, wherein the shell comprises a hydrophilic polymer.

25. The bone substitute of claim 19, wherein the shell comprises hydroxyethylmethacrylate, polyhydroxyethylmethacrylate, poly(ethylene glycol) methacrylate, poly(ethylene glycol)diacrylate, or a combination thereof.

26. The bone substitute of claim 19, wherein the shell is crosslinked using a photoinitiator blue dental light or a UV light.

27. The bone substitute of claim 19, further comprising a bone or soft tissue growth factor or a therapeutic agent.

28. A method of promoting bone generation comprising the steps:
(i) mixing a core comprising one or more first biologically-compatible material(s), a shell material comprising one or more second biologically-compatible hydrophilic polymer or polymerizable material(s) having at least one crosslinkable reactive group, and an initiator to form a crosslinkable bone substitute;
(ii) applying the crosslinkable bone substitute to an area in need of bone generation; and
(iii) crosslinking the bone substitute,
wherein the bone substitute promotes and/or induces bone generation.

29. The method of claim 28, wherein the initiator comprises a photoinitiator and crosslinking comprises applying light.

30. The method of claim 28, wherein the initiator comprises a redox couple.

31. The method of claim 28, wherein the initiator comprises component A which comprises a photochemical initiator and a radical generator; and component B which comprises a photochemical accelerator and a reducing agent.

32. A delivery system comprising:
(i) micron sized core particles comprising one or more first biologically-compatible material(s) and
(ii) a shell material comprising one or more second biologically-compatible hydrophilic polymer or polymerizable material(s) having at least one crosslinkable reactive group;
(iii) initiator component A comprising a photochemical initiator; and
(iv) initiator component B comprising a photochemical accelerator.

33. The delivery system of claim 32, wherein the shell material generally surrounds the core particles.

34. The delivery system of claim 32, wherein initiator component A further comprises an oxidizing agent and component B further comprises a reducing agent.

35. The delivery system of claim 32, wherein the core particles, shell material, and initiator component B are combined in one container and initiator component A is in a second container.

36. The delivery system of claim 32, wherein the photochemical initiator of component A is camphorquinone.

37. The delivery system of claim 32, wherein the oxidizing agent of component A is a peroxide or azo compound.

38. The delivery system of claim 32, wherein initiator component A comprises camphorquinone and benzoyl peroxide and initiator component B comprises 4-ethyl-dimethyl amino benzoate and N,N-dimethyl-p-toluidine.
